(19) United States
(12) Patent Application Publication
(10) Pub. No.: US 2025/0242165 A1
(43) Pub. Date: Jul. 31, 2025

(54) SYSTEMS AND METHODS FOR DETERMINING A PREDICTED DEVICE RUN TIME IN IMPLANTED MEDICAL DEVICES

(71) Applicant: Medtronic, Inc., Minneapolis, MN (US)

(72) Inventors: Andrew Thomas FRIED, St. Paul, MN (US); Emily G. KLOTSCHE, St. Paul, MN (US); Kunal J. PARALIKAR, Roseville, MN (US); Bhaskar RAVISHANKAR, Plymouth, MN (US); Bailey C. DERGAN, New Brighton, MN (US)

(21) Appl. No.: 18/990,837

(22) Filed: Dec. 20, 2024

Related U.S. Application Data
(60) Provisional application No. 63/625,808, filed on Jan. 26, 2024.

(51) Int. Cl. A61N 1/378; G16H 40/63

(52) U.S. Cl. CPC A61N 1/378; G16H 40/63

(57) ABSTRACT

An example system includes processing circuitry configured to determine a measured state of charge from a battery of an implantable medical device. The processing circuitry is configured to receive information relating to an average predicted current drain. The information includes one or more average predicted current drain parameters. The one or more average predicted current drain parameters include one or more of a current amplitude, a current pulse width, a current rate, a scheduled therapy duration, and a scheduled therapy session duty cycle. The processing circuitry is further configured to determine, based on the one or more average predicted current drain parameters, the average predicted current drain using a consumption model; determine, based on the measured state of charge and the average predicted current drain, a predicted depletion time of the battery; and generate, for output to a user, the predicted depletion time of the device.

### CROSS REFERENCE TO RELATED APPLICATIONS

This application claims priority to U.S. Provisional Patent Application No. 63/625,808, filed on Jan. 26, 2024, the contents of which are incorporated herein by reference in their entirety.

### FIELD

The disclosure relates to implantable medical devices, and, more specifically, systems and methods for monitoring a charge of a battery within implantable medical device.

### BACKGROUND

Medical devices may be external or implanted and may be used to monitor patient signals such as cardiac activity, biological impedance and to deliver electrical stimulation therapy to patients via various tissue sites to treat a variety of symptoms or conditions such as chronic pain, tremor, Parkinson's disease, epilepsy, urinary or fecal incontinence, sexual dysfunction, obesity, or gastroparesis and other conditions. In some examples, medical devices may include a rechargeable or primary cell electrical power source, or may be powered directly by transmitting energy through tissue.

### SUMMARY

In general, this disclosure is directed to devices, systems, and techniques for determining a predicted depletion time of a power source, such as a battery, in an implantable medical device (IMD). This disclosure is also directed to devices, systems, and techniques for using one or more methods of determining the predicted depletion time based on a measured state of charge of the battery and an average predicted current drain of the battery using a consumption model. In certain embodiments, the consumption model is an empirical model that may include a machine learning algorithm. This predicted depletion time of the battery can be used by the system to predict when recharging or replacement of the battery will be needed.

In certain examples, patients may not have a means for wirelessly recharging a battery within an implanted medical device (IMD). Some current systems only provide an estimated battery depletion time to the patient via a verbal conversation with the clinician at the time of follow up appointments and via an IMD battery percentage display. This may require patients to mentally estimate the depletion time based on the current battery depletion time and remaining therapy sessions required. It is desirable to provide an accurate battery depletion time to patients and clinicians that accounts for the current measured state of charge of the battery and a predicted current drain based on one or more predicted current drain parameters (e.g., an average current drain parameter).

The IMD or another device may determine the battery status of the IMD using a measured state of charge of the battery and an average predicted current drain that is determined using one or more average predicted current parameters such as the current amplitude (A), a current pulse width (P), a current rate (R), a scheduled therapy duration (D), and a scheduled therapy session duty cycle (X). The battery status may include an indication of at least one of an amount (e.g., a percentage) of remaining charge, a time until recharge, a recharge interval, an expected date of battery depletion, or an expected date of battery recharge. The IMD may be configured to generate information or a notification regarding the battery status. The notification regarding the battery status may be provided through a user interface of an external device, from the IMD itself (e.g., a unique stimulation pattern or device vibration), or another suitable method.

Due to the combination of average predicted current drain parameters and the estimated current drain for different periods of time or different device states, the IMD may provide a relatively accurate determination of a predicted device run time. Because the time between battery recharge may be long (e.g., on the order of days, months, or years, or longer), the use of both measured state of charge of the battery and the average predicted current drain (or some other value associated with battery usage) may provide a relatively accurate estimate of the predicted device run time for the device or user, and may facilitate planning for when recharge is needed. Further, because current drain of the battery of the IMD may vary between different patients (e.g., because of different therapy schedules) or devices, a suitably accurate determination of the battery status using this combination of battery usage techniques may better inform a user of the battery status, and facilitate planning for when recharge is needed.

In one example, a system includes processing circuitry configured to determine a measured state of charge from a battery of an implantable medical device. The processing circuitry is configured to receive information relating to an average predicted current drain. The information includes one or more average predicted current drain parameters. The one or more average predicted current drain parameters include one or more of a current amplitude, a current pulse width, a current rate, a scheduled therapy duration, and a scheduled therapy session duty cycle. The processing circuitry is further configured to determine, based on the one or more average predicted current drain parameters, the average predicted current drain using a consumption model; determine, based on the measured state of charge and the average predicted current drain, a predicted depletion time of the battery; and generate, for output to a user, the predicted depletion time of the device.

In another example, a method includes determining a measured state of charge from a battery of an implantable medical device. Furthermore, the method includes receiving information relating to an average predicted current drain (I_drain), wherein the information includes one or more average predicted current drain parameters, wherein the one or more average predicted current drain parameters include one or more of a current amplitude (A), a current pulse width (P), a current rate (R), a scheduled therapy duration (D), and a scheduled therapy session duty cycle (X). Further yet, the method includes determining, based on the one or more average predicted current drain parameters, the average predicted current drain using an empirical system model. Still further yet, the method includes determining, based on the measured state of charge and the average predicted current drain, a predicted device run time of the battery. Still further yet, the method includes generating, for output to a user, the predicted device run time of the battery.

In another example, a method for training a machine learning algorithm to predict a depletion time of a battery within an implantable medical device (IMD) includes providing a labeled dataset corresponding to one or more IMDs, wherein the labeled dataset includes, for each IMD: a measured state of charge from the battery; one or more average predicted current drain parameters comprising a current amplitude (A), a current pulse width (P), a current rate (R), a scheduled therapy duration (D), and a scheduled therapy session duty cycle (X); and a labeled depletion time of the battery. Furthermore, the method includes determining, based on the measured state of charge from the battery and the one or more average predicted current drain parameters, an average predicted current drain of one or more batteries. Further yet, the method includes determining, based on the measured state of charge and the average predicted current drain, an estimated run time of the battery. Still further yet, the method includes evaluating the machine learning algorithm's performance on the labeled dataset by comparing the estimated run time of the battery to the labeled depletion time of the battery. Still further yet, the method includes repeating one or more of the above steps until a desired level of accuracy is achieved.

The above summary is not intended to describe each illustrated embodiment or every implementation of the subject matter hereof. The figures and the detailed description that follow more particularly exemplify various embodiments.

### BRIEF DESCRIPTION OF DRAWINGS

Subject matter hereof may be more completely understood in consideration of the following detailed description of various embodiments in connection with the accompanying figures, in which:

FIG. 1 is a conceptual diagram illustrating a leg having an IMD, such as a leadless neurostimulation device, implanted near a tibial nerve of a patient, in accordance with one or more embodiments of the present disclosure.

FIG. 2 is a block diagram illustrating example components of the implantable medical device of FIG. 1, in accordance with one or more embodiments of the present disclosure.

FIG. 3 is a block diagram of an example charging device of FIG. 1, in accordance with one or more embodiments of the present disclosure.

FIG. 4 is a is a block diagram of an example programmer of FIG. 1, in accordance with one or more embodiments of the present disclosure.

FIG. 5 is a block diagram illustrating example system inputs that are received by the programmer of FIGS. 1 and 4, in accordance with one or more embodiments of the present disclosure.

FIG. 6 is a block diagram illustrating example system outputs generated by the programmer of FIGS. 1 and 5, in accordance with one or more embodiments of the present disclosure.

FIG. 7 is a scatter plot diagram measuring an actual battery current vs. a programmed stimulation current using the system of FIG. 1, in accordance with one or more embodiments of the present disclosure.

FIG. 8 is a scatter plot diagram measuring an actual current drain of a battery within an IMD vs. an estimated current drain of the battery within the IMD using the system of FIG. 1, in accordance with one or more embodiments of the present disclosure.

FIG. 9 is a diagram measuring a programmed stimulation amplitude of a battery within an IMD at different time intervals including post-operative programmed stimulation amplitude, a programmed stimulation amplitude measured seven days from implantation, a programmed stimulation amplitude measured fourteen days from implantation, a programmed stimulation amplitude measured one month from implantation, a programmed stimulation amplitude measured three months from implantation, and a programmed stimulation amplitude measured six months from implantation, in accordance with one or more embodiments of the present disclosure.

FIG. 10 is a diagram measuring the impedance of a battery within an IMD at different time intervals including a post-operative impedance measurement, an impedance measurement seven days from implantation, an impedance measurement fourteen days from implantation, an impedance measurement one month from implantation, an impedance measurement three months from implantation, and an impedance measurement six months from implantation, in accordance with one or more embodiments of the present disclosure.

FIG. 11 is a diagram measuring an estimated current drain of a battery within an IMD at different time intervals including post-operative estimated battery current, an estimated battery current seven days from implantation, an estimated battery current fourteen days from implantation, an estimated battery current one month from implantation, an estimated battery current three months from implantation, and an estimated battery current six months from implantation, in accordance with one or more embodiments of the present disclosure.

FIG. 12 is a flow chart diagram of a method for determining a predicted device run time of a battery and generating an output to a user including the predicted device run time, in accordance with one or more embodiments of the present disclosure.

FIG. 13 is a flow chart diagram of a method for training a machine learning algorithm to predict a depletion time of a battery within an IMD, in accordance with one or more embodiments of the present disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

This disclosure describes devices, systems, and techniques for determining a predicted depletion time of a power source, such as a rechargeable or replaceable battery in an implantable medical device (IMD).

The status of the power source (e.g., the battery status) of the IMD may inform a user of when recharging of the power source (e.g., the battery) is needed. In some examples, the IMD is configured to provide information indicative of the status of power source, which may include an indication of at least one of an amount (e.g., a percentage) of remaining charge, a time until recharge, a recharge interval, an expected date of battery depletion, or an expected date of battery recharge. A suitably accurate determination of the battery status may better inform a user of the battery status and facilitate planning for when recharge is needed.

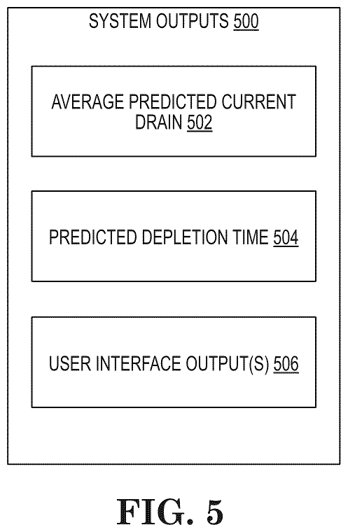

In certain examples, the status of the power source of the IMD may be determined using a model (e.g., an empirical model or machine learning algorithm). As described herein, the model can consider one or more characteristics of IMD and a therapy schedule that is administered to a patient. Inputs for the consumption model are illustrated and described in further detail with respect to FIG. 5.

Although certain embodiments of the devices, systems, and techniques described herein are described in the context of estimating a status of a power source (e.g., a battery status) of IMDs with rechargeable batteries for a tibial nerve stimulator configured to provide tibial nerve stimulation, the techniques described herein may be applicable to other devices configured for other types of therapy. For example, the techniques of this disclosure may be applicable for other types of devices configured for invasive or noninvasive neuromodulation for pain relief, muscle activation, or other therapeutic benefits. Additionally, the techniques of this disclosure are not limited to rechargeable power sources but are also applicable to other types of power sources (e.g., non-rechargeable power sources, primary cell, etc.).

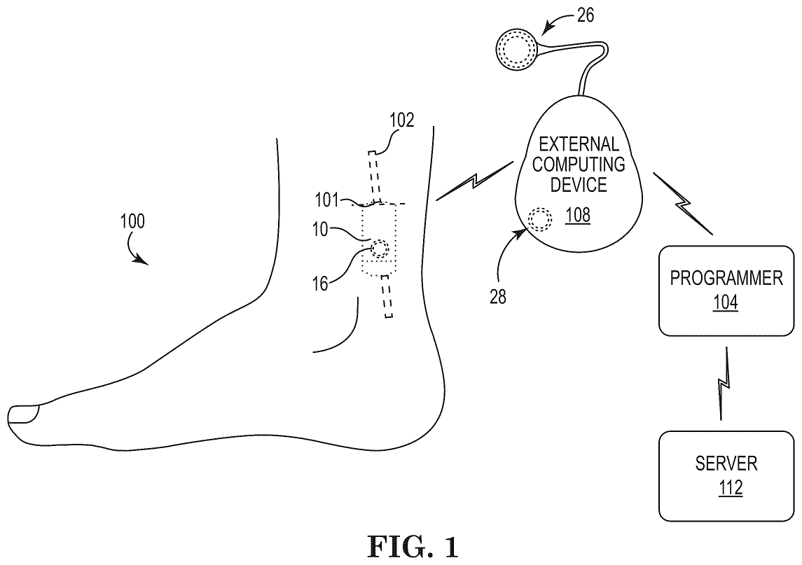

FIG. 1 is a conceptual diagram illustrating a leg having an IMD 10, such as a leadless neurostimulation device, implanted near a tibial nerve. Although a leadless neurostimulation is shown by illustrative example, the systems and methods described herein are not limited to implantable medical devices that are shown. The example of system 100 in FIG. 1 includes an implantable medical device (IMD) 10, an external computing device 108, a programmer 104 (which may be a patient programmer or a clinician programmer), and a server 112. In other examples, the techniques of this disclosure may be implemented in other battery-powered devices, for example, an implantable drug pump.

External computing device 108 can include one or more charging coils, such as external primary coil 26 or internal primary coil 28. External computing device 108 may be used to program or adjust settings of IMD 10 and may also recharge an electrical energy storage device, such as a battery, of IMD 10. External computing device 108 may also communicate with server 112. In other examples, an external device (e.g., programmer 104) separate from external computing device 108 may communicate with IMD 10 to adjust therapy or sensing parameters, download recorded data, or perform other functions.

Server 112 may be one or more servers in a local network or in a cloud computing environment. Server 112 may be configured to communicate with programmer 104, external computing device 108 or IMD 10 via wireless communication through a network access point (not shown in FIG. 1) and may be co-located with external computing device 108 or programmer 104, or may be located elsewhere, such as in a cloud computing data center.

The example of FIG. 1 is a side view of a patient's leg showing a leadless neurostimulation IMD 10 near the ankle adjacent to the tibial nerve 102. IMD 10 can be implanted through the patient's skin and cutaneous fat layer via a small incision 101 (e.g., about one to three centimeters (cm)) above the tibial nerve on a medial aspect of the patient's ankle. While incision 101 is shown approximately horizontal to the length of the tibial nerve, other incisions or implantation techniques could be used according to physician preference. The example of FIG. 1 describes a neurostimulation implantable medical device for tibial nerve stimulation. In other examples, the techniques of this disclosure may apply to other devices, such as implantable neurostimulation system for use in spinal cord stimulation therapy and deep brain stimulation, as well as to other types of medical devices without limitation.

IMD 10 may be constructed of any polymer, metal, or composite material sufficient to house the components of IMD 10. In some examples, IMD 10 is constructed with a biocompatible housing, such as titanium or stainless steel, or a polymeric material such as silicone or polyurethane, and surgically implanted at a site in patient near the tibial nerve. In other examples, IMD 10 is implanted near the pelvis, abdomen, or buttocks. The housing of IMD 10 may be configured to provide a hermetic seal for components, such as a rechargeable power source. In addition, the housing of IMD 10 may be selected of a material that facilitates receiving energy to charge the rechargeable power source.

During operation, an electrical stimulation signal may be transmitted between one or more electrodes through the fascia layer. The electrical signal may be used to stimulate tibial nerve 102 which may be useful in the treatment of overactive bladder (OAB) symptoms of urinary urgency, urinary frequency or urge incontinence, fecal incontinence, pain or other symptoms.

In some examples, disease, age, and injury may impair physiological functions of a patient. In one example, an IMD 10 can be used to treat patients with bladder dysfunction. Bladder dysfunction, such as overactive bladder, urgency, or urinary incontinence, is a problem that may afflict people of all ages, genders, and races. Various muscles, nerves, organs, and conduits within the pelvic floor cooperate to collect, store and release urine. A variety of disorders may compromise urinary tract performance, and contribute to an overactive bladder, urgency, or urinary incontinence that interferes with normal physiological function. System 100 may help relieve some symptoms of some disorders.

The rechargeable power source of IMD 10 may include one or more capacitors, batteries, or other components (e.g., chemical or electrical energy storage devices). Example batteries may include lithium-based batteries, nickel metal-hydride batteries, or other materials. The rechargeable power source may be replenished, refilled, or otherwise capable of increasing the amount of energy stored after energy has been depleted. The energy received from secondary coil 16 may be conditioned or transformed by a charging circuit. The charging circuit may then send an electrical signal used to charge the rechargeable power source when the power source is fully depleted or only partially depleted.

External computing device 108 may be used to recharge the rechargeable power source within IMD 10 implanted in the patient. External computing device 108 may be a hand-held device, a portable device, or a stationary charging system. External computing device 108 may also be referred to as charging device 108 in this disclosure. External computing device 108 may include components necessary to charge IMD 10 through tissue of the patient. External computing device 108 may include an internal primary coil 28 and external primary coil 26. In other examples, external computing device may only include internal primary coil 28 and omit the use of external primary coil 26, or only include external primary coil 26 and omit the use of internal primary coil 28. External computing device 108 may include a housing to enclose operational components such as a processor, memory, user interface, telemetry module, power source, and charging circuit configured to transmit energy to secondary coil 16 via external primary coil 26 or internal primary coil 28. Although a user may control the recharging process with a user interface of external computing device 108, external computing device 108 may alternatively be controlled by another device, e.g., programmer 104, a computing device of server 112 such as a tablet computer, laptop or other similar computing device. The second external computing device of server 112 may include a computing device with a touch-screen user interface. In other examples, external computing device 108 may be integrated with an external programmer, such as patient programmer 104, which may be carried by the patient.

External computing device 108 and IMD 10 may utilize any wireless power transfer techniques that are capable of recharging the power source of IMD 10 when IMD 10 is implanted within the patient. In some examples, system 100 may utilize inductive coupling between internal primary coil 28 or external primary coil 26 of external computing device 108 and secondary coils (e.g., secondary coil 16) of IMD 10. In inductive coupling, internal primary coil 28 is placed near implanted IMD 10 such that internal primary coil 28 is aligned with secondary coil 16 of IMD 10. External computing device 108 may then generate an electrical current in internal primary coil 28 based on a selected power level for charging the rechargeable power source of IMD 10. When either internal primary coil 28 or external primary coil 26 are aligned with secondary coil 16, the electrical current in either internal primary coil 28 or external primary coil 26 may magnetically induce an electrical current in secondary coil 16 within IMD 10. Since secondary coil 16 is associated with and electrically coupled to the rechargeable power source, the induced electrical current may be used to increase the voltage, or charge level, of the rechargeable power source. Although inductive coupling is generally described herein, any type of wireless energy transfer may be used to transfer energy between external computing device 108 and IMD 10.

External primary coil 26 or internal primary coil 28 may include a wound wire (e.g., a coil) (not shown in FIG. 1). The coil may be constructed of a wire wound in an in-plane spiral (e.g., a disk-shaped coil). In some examples, this single or even multi-layers spiral of wire may be considered a flexible coil capable of deforming to conform with a non-planar skin surface. The coil may include wires that electrically couple the flexible coil to a power source and a charging module configured to generate an electrical current within the coil. Internal primary coil 28 may be external of the housing of external computing device 108 such that internal primary coil 28 can be placed on the skin of the patient proximal to IMD 10. In some examples, internal primary coil 28 may be disposed on the outside of the housing or even within housing.

Either external primary coil 26 or internal primary coil 28 of system 100 may include a heat sink device (not shown in FIG. 1). In the example of system 100, external computing device 108 is the power transmitting unit and IMD 10 is the power receiving unit. IMD 10 may be in a flipped or non-flipped position.

As noted above, external computing device 108 may also be referred to as recharger 108. Recharger 108 may include a user interface to receive control inputs from a user, such as the patient, medical professional or other caregiver. The user interface of recharger 108 may also provide information to a user. For example, recharger 108 may include a control configured to receive user input (not shown in FIG. 1) as well as a set of indicator lights. In some examples the indicator lights may be configured to illuminate the control. The indicator lights may also be configured to output information regarding an operational state of external computing device 108, such as a communication status and wireless power transfer status.

The processing circuitry determines whether IMD 10 and recharger 108 have established a communication link e.g., via communication circuitry. In response to the processing circuitry determining that recharger 108 and IMD 10 have not established a communication link, the processing circuitry may cause a notification to be generated. Recharger 108 may still wirelessly transfer power to IMD 10, but the notification may signify that recharger 108 is operating in open loop charging mode.

Processing circuitry of system 100, e.g., processing circuitry of recharger 108, processing circuitry of server 112, or processing circuitry of IMD 10, may calculate any of the values described herein.

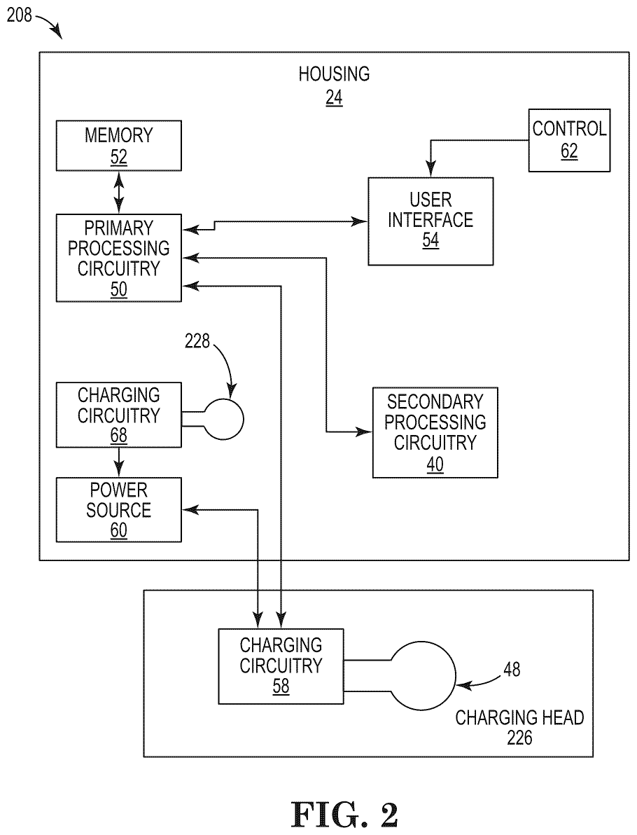

FIG. 2 is a block diagram illustrating example components of the medical device of FIG. 1. Implantable medical device (IMD) 210 is an example of IMD 10 described above in relation to FIG. 1. In the example illustrated in FIG. 2, IMD housing 19 of IMD 210 encloses temperature sensor 39, secondary coil 16, processing circuitry 30, therapy generation and sensing circuitry 34, recharge circuitry 38, memory 32, telemetry circuitry 36, power source 18, switch 33, coulomb counter 35, state control circuitry 31, and, in some examples, one or more sensors 37, such as an accelerometer. In other examples, IMD 210 may include a greater or a fewer number of components, e.g., in some examples, IMD 210 may not include temperature sensor 39 or sensors 37. In general, IMD 210 may comprise any suitable arrangement of hardware, alone or in combination with software or firmware, to perform the various techniques described herein attributed to IMD 210 and processing circuitry 30, and any equivalents thereof.

Processing circuitry 30 of IMD 210 may include one or more processors, such as one or more microprocessors, digital signal processors (DSPs), application specific integrated circuits (ASICs), field programmable gate arrays (FPGAs), or any other equivalent integrated or discrete logic circuitry, as well as any combinations of such components. IMD 210 may include a memory 32, such as random access memory (RAM), read only memory (ROM), programmable read only memory (PROM), erasable programmable read only memory (EPROM), electronically erasable programmable read only memory (EEPROM), flash memory, comprising executable instructions for causing the processing circuitry 30 to perform the actions attributed to this circuitry. Moreover, although processing circuitry 30, therapy generation and sensing circuitry 34, recharge circuitry 38, telemetry circuitry 36, temperature sensor 39, state control circuitry 31, coulomb counter 35, switch 33, and timer 41 are described as separate modules, in some examples, some combination of processing circuitry 30, therapy generation and sensing circuitry 34, recharge circuitry 38, telemetry circuitry 36, temperature sensor 39, state control circuitry 31, coulomb counter 35, and switch 33 are functionally integrated. In some examples, processing circuitry 30, therapy generation and sensing circuitry 34, recharge circuitry 38, telemetry circuitry 36, and temperature sensor 39, state control circuitry 31, coulomb counter 35, and switch 33 correspond to individual hardware units, such as ASICs, DSPs, FPGAs, or other hardware units. In this disclosure, therapy generation and sensing circuitry 34 may be referred to as therapy generation circuitry 34, for simplicity.

Memory 32 may store therapy programs or other instructions that specify therapy parameter values for the therapy provided by therapy generation circuitry 34 and IMD 210. In some examples, memory 32 may also store temperature data from temperature sensor 39, instructions for recharging rechargeable power source 18, thresholds, instructions for communication between IMD 210 and an external computing device, or any other instructions required to perform tasks attributed to IMD 210. Memory 32 may be configured to store instructions for communication with or controlling one or more temperature sensors of temperature sensor 39. In various examples, memory 32 stores information related to determining the temperature of housing 19 or exterior surface(s) of housing 19 of IMD 210 based on temperatures sensed by one or more temperature sensors, such as temperature sensor 39, located within IMD 210.

For example, memory 32 may store programming settings such as electrical stimulation therapy output magnitude, pulse width, and so on. Memory 32 may determine whether a sensed bioelectrical signal is valid, such as an evoked compound action potential (ECAP) or other signal in response to an output electrical stimulation therapy event. Memory 32 may store programming instructions that when executed by processing circuitry 30 cause processing circuitry 30 to cause therapy generation circuitry 34 to deliver electrical stimulation therapy to a target nerve of a patient.

Therapy generation and sensing circuitry 34 may generate and deliver electrical stimulation under the control of processing circuitry 30. In some examples, processing circuitry 30 controls therapy generation circuitry 34 by accessing memory 32 to selectively access and load at least one of the stimulation programs to therapy generation circuitry 34. For example, in operation, processing circuitry 30 may access memory 32 to load one of the stimulation programs to therapy generation circuitry 34. In such examples, relevant stimulation parameters may include a voltage amplitude, a current amplitude, a pulse rate, a pulse width, a duty cycle, or the combination of electrodes 17A, 17B, 17C, and 17D (collectively “electrodes 17”) that therapy generation circuitry 34 may use to deliver the electrical stimulation signal as well as sense biological signals. In other examples, IMD 210 may have more or fewer electrodes than the four shown in the example of FIG. 2. In some examples, electrodes 17 may be part of or attached to a housing of IMD 210, e.g., a leadless electrode. In other examples, one or more of electrodes 17 may be part of a lead implanted in or attached to a patient to sense biological signals or deliver electrical stimulation, as described above in relation to FIG. 1.

In some examples, one or more electrodes 17 connected to therapy generation circuitry 34 may connect to one or more sensing electrodes, e.g., attached to housing of IMD 210. In some examples, electrodes 17 may be configured to detect the evoked motor response caused by the electrical stimulation therapy event, or other bioelectrical signals such as ECAPs, impedance and so on.

IMD 210 also includes components to receive power to recharge rechargeable power source 18 when rechargeable power source 18 has been at least partially depleted. As shown in FIG. 2, IMD 210 includes secondary coil 16 and recharge circuitry 38 coupled to rechargeable power source 18. Recharge circuitry 38 may be configured to charge rechargeable power source 18 with the selected power level determined by either processing circuitry 30 or an external charging device, such as external computing device 108 described above in relation to FIG. 1. Recharge circuitry 38 may include any of a variety of charging or control circuitry configured to process or convert current induced in secondary coil 16 into charging current to charge power source 18.

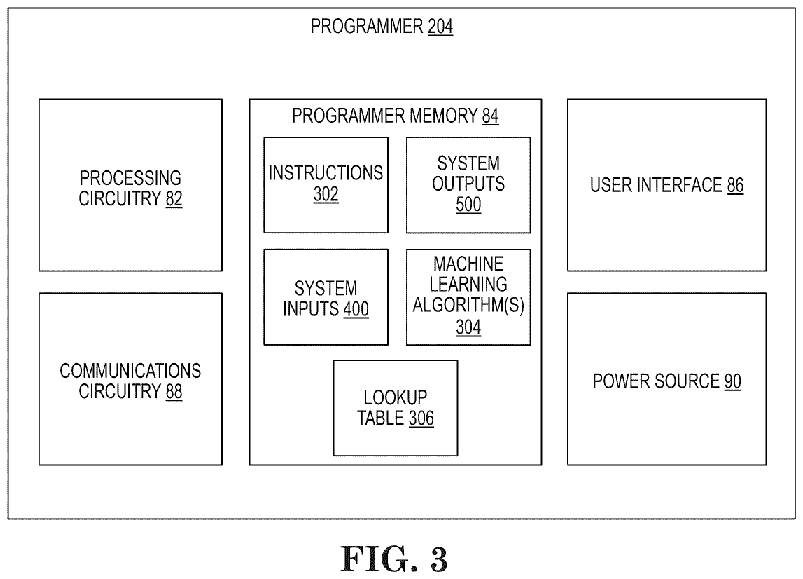

As described herein with reference to FIGS. 3-11 below, The IMD 210 may be recharged using the rechargeable power source 18 at a desirable time as identified by a model (such as an empirical recharge model). The model may take into account several inputs that are illustrated and described in further detail with respect to FIG. 5.

Secondary coil 16 may include a coil of wire or other device capable of inductive coupling with a primary coil disposed external to a patient. Although secondary coil 16 is illustrated as a simple loop in FIG. 2, secondary coil 16 may include multiple turns of conductive wire. Secondary coil 16 may include a winding of wire configured such that an electrical current can be induced within secondary coil 16 from a magnetic field. The induced electrical current may then be used to recharge rechargeable power source 18.

Recharge circuitry 38 may include one or more circuits that process, filter, convert or transform the electrical signal induced in the secondary coil to an electrical signal capable of recharging rechargeable power source 18. For example, in alternating current induction, recharge circuitry 38 may include a half-wave rectifier circuit or a full-wave rectifier circuit configured to convert alternating current from the induction to a direct current for rechargeable power source 18. The full-wave rectifier circuit may be more efficient at converting the induced energy for rechargeable power source 18. However, a half-wave rectifier circuit may be used to store energy in rechargeable power source 18 at a slower rate. In some examples, recharge circuitry 38 may include both a full-wave rectifier circuit and a half-wave rectifier circuit such that recharge circuitry 38 may switch between each circuit to control the charging rate of rechargeable power source 18 and temperature of IMD 210.

Rechargeable power source 18 may include one or more capacitors, batteries, or other energy storage devices. Rechargeable power source 18 may deliver operating power to the components of IMD 210. In some examples, rechargeable power source 18 may include a power generation circuit to produce the operating power. Rechargeable power source 18 may be configured to operate through many discharge and recharge cycles. Rechargeable power source 18 may also be configured to provide operational power to IMD 210 during the recharge process. In some examples, rechargeable power source 18 may be constructed with materials to reduce the amount of heat generated during charging. In other examples, IMD 210 may be constructed of materials or using structures that may help dissipate generated heat at rechargeable power source 18, recharge circuitry 38, or secondary coil 16 over a larger surface area of the housing of IMD 210. In some examples, power source 18 includes a non-rechargeable power source.

Although rechargeable power source 18, recharge circuitry 38, and secondary coil 16 are shown as contained within the housing of IMD 210, in alternative implementations, at least one of these components may be disposed outside of the housing. For example, in some implementations, secondary coil 16 may be disposed outside of the housing of IMD 210 to facilitate better coupling between secondary coil 16 and the primary coil of external charging device. In other examples, power source 18 may be a primary power cell and IMD 210 may not include recharge circuitry 38 and secondary coil 16.

Processing circuitry 30 may also control the exchange of information with an external computing device using telemetry circuitry 36. Telemetry circuitry 36 may be configured for wireless communication using radio frequency (RF) protocols, such as Bluetooth, including Bluetooth low energy (BLE), or similar RF protocols, as well as using inductive communication protocols. Telemetry circuitry 36 may include one or more antennas configured to communicate with an external charging device (e.g., external computing device 108 of FIG. 1). Processing circuitry 30 may transmit operational information and receive therapy programs or therapy parameter adjustments via telemetry circuitry 36. Also, in some examples, IMD 210 may communicate with other implanted devices, such as stimulators, control devices, or sensors, via telemetry circuitry 36. In addition, telemetry circuitry 36 may be configured to control the exchange of information related to sensed or determined temperature data, for example temperatures sensed by or determined from temperatures sensed using temperature sensor 39. In some examples, telemetry circuitry 36 may communicate using inductive communication, and in other examples, telemetry circuitry 36 may communicate using RF frequencies separate from the frequencies used for inductive charging.

In some examples, processing circuitry 30 may transmit, via control of telemetry circuitry 36, additional information to external charging device related to the operation of rechargeable power source 18. For example, processing circuitry 30 may control telemetry circuitry 36 to transmit indications that rechargeable power source 18 is completely charged, rechargeable power source 18 is fully discharged, the amount of charging current output by recharge circuitry 38 e.g., to power source 18, or any other charge status of rechargeable power source 18. In some examples, processing circuitry 30 may use telemetry circuitry 36 to transmit instructions to external charging device, including instructions regarding further control of the charging session, for example instructions to lower the power level or to terminate the charging session, based on the determined temperature of IMD housing 19.

Processing circuitry 30 may also transmit information to external charging device that indicates any problems or errors with rechargeable power source 18 that may prevent rechargeable power source 18 from providing operational power to the components of IMD 210. In various examples, processing circuitry 30 may receive, through telemetry circuitry 36, instructions for algorithms, including formulas or values for constants to be used in the formulas, that may be used to determine the temperature of the housing 19 or exterior surface(s) of housing 19 of IMD 210 based on temperatures sensed by temperature sensor 39 located within IMD 210 during and after a recharging session performed on rechargeable power source 18.

IMD 210 also includes components for determining a status of power source 18. For example, in examples where power source 18 includes a battery, IMD 210 may include components for determining (e.g., measuring, estimating, receiving, etc.) information related to a battery status or battery charge or activity (e.g., an amount of current drain from the battery, an amount of charge remaining in the battery, etc.). Components of IMD 210 for determining information related to the battery status include coulomb counter 35, switch 33, timer 41, and state control circuitry 31. Coulomb counter 35, switch 33, timer 41, and state control circuitry 31 may be used alone or in connection with other components of IMD 210, including processing circuitry 30.

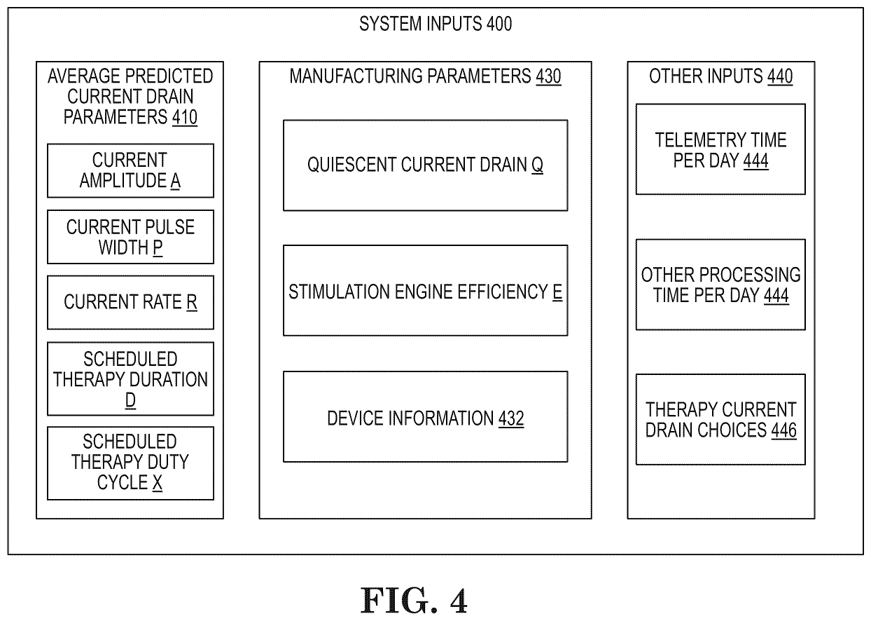

In some examples, IMD 210 uses one or more methods or combinations of components to determine the status of power source 18 of IMD 210. These methods and combinations are described in further detail with respect to FIGS. 4-6 and 12-13.

In some examples, coulomb counter 35 is be configured to measure current drain from power source 18 (e.g., a battery) of IMD 210. In certain examples, coulomb counter 35 may output an average current or cumulative current over a period of time to processing circuitry 30 for determining the current drain during a period of time. Because coulomb counter 35 directly measures current drain from power source 18 (e.g., a battery), the measured current may be more accurate as compared to other methods of determining current drain (e.g., methods involving indirect measurement or estimation of current drain). Processing circuitry 30 may be configured to receive the measured current drain, or one or more values indicative of the measure current drain, from coulomb counter 35.

In some examples, processing circuitry 30 is configured to determine the current drain from power source 18 by using, or at least partially using, one or more stored values (e.g., via a lookup table). For example, processing circuitry 30 may be configured to access (via memory 32, programmer 104, or server 112 etc.) one or more stored values related to or indicative of the average predicted current drain parameters. As further discussed in FIG. 4 below, the one or more stored values (e.g., those accessed via a lookup table) can include a current amplitude A, a current pulse width P, and a current rate R. Furthermore, in certain examples, the current amplitude A, a current pulse width P, and a current rate R can be stored in a three-dimensional lookup table with the values being stored on an X axis, a Y axis, and a Z axis.

In addition, or alternative, to the methods of determining a current drain from power source 18 or a status of power source 18 noted above, IMD 210, via processing circuitry 30, may be configured to measure a voltage of power source 18 (e.g., a battery) as part of determining the battery status or battery usages.

IMD 210, via processing circuitry 30, may be configured to generate, for output, information indicative of the status of power source 18 (e.g., a battery status, in examples where power source 18 includes at least a battery). Information indicative of the status of power source 18 (e.g., a battery status) may include an indication of at least one of an amount (e.g., a percentage) of remaining charge, a time until recharge, a recharge interval, a date of charge depletion, an expected date of battery depletion, or an expected date of battery recharge. In some examples, IMD 210, via processing circuitry 30, may be configured generate for output information indicative of the status of power source 18 at pre-determined events (e.g., battery percentage thresholds of remaining charge, such as 20 percent, 10 percent, etc.). The information indicative of the status of power source 18 may be updated automatically on a periodic basis, after one or more events (e.g., a recharge session, the start or end of a therapy session, etc.), or upon interrogation of IMD 210 by an external device (e.g., programmer 104, external computing device 108, or server 112).

Information indicative of the status of power source 18 (e.g., a battery status) may be generated from a model using one or more inputs. For example, a processing circuitry 30 may predict or estimate a recharge interval based on a model. In some examples, information indicative of the status of power source 18 incorporates information of past use or predicted future use of IMD 210. For example, IMD 210 may be configured to access one or more therapy schedules or other predicted future use to determine (e.g., estimate or predict) the status of power source 18. In this way, IMD 210 may also use past current drain (e.g., measured or estimated) as well as expected future current drain (e.g., from a therapy schedule) to indicate a time until recharge is needed, a recharge interval, a date of charge depletion, an expected date of battery depletion, or an expected date of when battery recharge is needed. As discussed previously, IMD 210 may be configured to use recharge session information (e.g., amount of time spent in a recharge session or amount of charge gained during a recharge session) to generate information indicative of the status of power source 18. Inputs for the model are discussed in further detail below with respect to FIG. 5.

In some examples, notifications regarding the status of power source 18 may inform a user of information indicative of the status of power source 18 (e.g., a battery status). For example, processing circuitry 30 may be configured to generate a notification when a charge of power source 18 drops below a predefined threshold, or when another user defined event has occurred. For example, a user may specify a time threshold since the last recharge session to cause processing circuitry 30 to provide a notification or prompt to recharge power source 18 after the time threshold has elapsed. Similarly, a user may specify a maximum recharge time to cause processing circuitry 30 to provide a notification or prompt to recharge once the time needed to recharge power source 18 reaches the user-specified maximum recharge time.

In some examples, processing circuitry 30 is configured to generate one or more of a visual, audio, tactile, haptic feedback, or related notification regarding the status of power source 18. Processing circuitry 30 may be configured to output a notification or information regarding the status of power source 18 via programmer 104 or external computing device 108 (e.g., via user interface 54 of external charging device 208 or via user interface 86 of programmer 204, shown in FIG. 3 and FIG. 4 respectively). In some examples, a notification or information regarding the status of power source 18 includes an estimated date of charge depletion and or an amount of time remaining until the charge level of power source 18 is depleted or depleted below a level needed for proper functioning of IMD 210. In some examples, a notification or information regarding the status of power source 18 includes one or more prompts (e.g., one-time, or at an interval) to a user to recharge power source 18. Processing circuitry 30 may be configured to provide a haptic feedback notification (e.g., a vibration) via IMD 210 while implanted in a patient to alert the patient of the status of power source 18. For example, processing circuitry 30 may be configured to cause IMD 210 to vibrate in a predetermined pattern of pulses to alert a patient of a low level of charge, prompt recharging, or provide other information related to the status of power source 18 (e.g., the battery). IMD 210 may vibrate before or after a stimulation session, or periodically (e.g., daily) to provide a notification related to the status of power source 18. Processing circuitry 30 may be configured to cause a IMD 210 to output a stimulation-based notification (e.g., a unique stimulation profile via electrodes 17 or stimulation at the subcutaneous pocket containing the housing of IMD 210) to alert the patient of the status of power source 18. For example, processing circuitry 30 may be configured to cause IMD 210 to generate a unique predetermined stimulation pattern to alert a patient of a low level of charge, prompt recharging, provide other information related to the status of power source 18 (e.g., the battery). In some examples, processing circuitry 30 may be configured to alert a patient, clinician, or other user of the status of power source 18 via programmer 104 (in examples where programmer 104 includes a clinician programmer), via external computing device 108, via server 112, or via another suitable method. Visual alerts may include where a light source on IMD 210 (e.g., a light-emitting diode (LED)) emits light to provide a notification related to the status of power source 18. Alerts regarding the status of power source 18 to a patient, clinician, or other user may additionally or alternatively include one or more of a text-message, automated phone call, email, or notification through a mobile-device application.

In examples where a stimulation-based notification (e.g., a unique stimulation pattern) is used to inform a user of the status of power source 18, one or more parameters may be configured by the clinician or the patient. The unique stimulation pattern may include intervals of stimulation, increasing or decreasing rates of pulses, or varying amplitudes of stimulation. As an example, processing circuitry 30 may be configured to cause IMD 210 to provide a stimulation pattern of 1 second on, 2 seconds off, then 1 second on, 1 second off, then 1 second on, 0.5 seconds off, followed by a ramp down after the scheduled therapy session is delivered. A patient may be trained to the unique stimulation pattern, or another source of information (e.g., a device label, or user interface) may inform a patient to be aware of the unique stimulation pattern (e.g., such as for recharging power source 18). In some examples, the clinician or the patient sets a perception threshold for a recharge reminder. In some examples, the clinician or patient can select whether or when the stimulation-based notification is given. For example, the clinician or patient may set the stimulation-based notification to occur before or after a stimulation session, or at another time. As another example, the clinician or patient may set the stimulation-based notification to occur on a unique schedule. Besides informing the user of the status of power source 18, a unique stimulation-based notification may be used to alert the patient to perform another task (e.g., open a device application, check the status of IMD 210, recharge power source 18, etc.).

FIG. 3 is a block diagram of an example an external computing device of FIG. 1. External charging device 208 in of FIG. 3 is an example of external computing device 108 described above in relation to FIG. 1. In some examples, external charging device 208 may be described as a hand-held device, in other examples, external charging device 208 may be a larger or a non-portable device. In addition, in other examples external charging device 208 may be included as part of an external programmer or include functionality of an external programmer. As shown in the example of FIG. 3, external charging device 208 includes a housing 24 connected to a charging head 226. Housing 24 encloses components such as a primary processing circuitry 50, memory 52, user interface 54, telemetry circuitry 56, control 62, one or more sets of indicator lights 64, audio output circuitry 70, haptic output circuitry 72 and power source 60. Charging head 226 may include charging circuitry 58, temperature sensor 59, and external primary coil 48. Charging head 226 or external primary coil 48 may be an example of external primary coil 26 as shown in FIG. 1. Housing 24 is electrically coupled to charging head 226 via a cable. Housing 24 may also include charging circuitry 68 and internal primary coil 228, which is an example of internal primary coil 28 described above in relation to FIG. 1.

In some examples, separate charging head 226 may facilitate positioning of external primary coil 48 over secondary coil 16 of IMD 10 (as shown in FIG. 1) or IMD 210 (as shown in FIG. 2). In some examples, charging circuitry 68 or internal primary coil 228 may be integrated within housing 24. In other examples, external charging device 208 may not include charging head 226. Memory 52 may store instructions that, when executed by primary processing circuitry 50, causes primary processing circuitry 50 and external charging device 208 to provide the functionality ascribed to external charging device 208 throughout this disclosure, or any equivalents thereof. External primary coil 48 and internal primary coil 228 may also be referred to as an antenna. In some examples, external charging device 208 may include secondary processing circuitry 40, which may control telemetry circuitry 56, as well as perform other functions. Some other functions may include error checking of the operation of primary processing circuitry 50.

External charging device 208 may also include one or more temperature sensors, illustrated as temperature sensor 59 within charging head 226, similar to temperature sensor 39 of FIG. 2. As shown in FIG. 3, temperature sensor 59 may be disposed within charging head 226. In other examples, one or more temperature sensors of temperature sensor 59 may be disposed within housing 24. For example, charging head 226 may include one or more temperature sensors positioned and configured to sense the temperature of external primary coil 48 or a surface of the housing of charging head 226. In some examples, external charging device 208 may not include temperature sensor 59.

In general, external charging device 208 comprises any suitable arrangement of hardware, alone or in combination with software or firmware, to perform the techniques ascribed to external charging device 208, and primary processing circuitry 50, user interface 54, telemetry circuitry 56, and charging circuitry 68 of external charging device 208, or any equivalents thereof. In various examples, external charging device 208 may include one or more processors, such as one or more microprocessors, DSPs, ASICS, FPGAs, or any other equivalent integrated or discrete logic circuitry, as well as any combinations of such components. External charging device 208 also, in various examples, may include a memory 52, such as RAM, ROM, PROM, EPROM, EEPROM, flash memory, a hard disk, a CD-ROM, comprising executable instructions for causing the one or more processors to perform the actions attributed to them. Moreover, although primary processing circuitry 50, telemetry circuitry 56, charging circuitry 68, and temperature sensor 59 are described as separate modules, in some examples, primary processing circuitry 50, telemetry circuitry 56, charging circuitry 68, or temperature sensor 59 are functionally integrated. In some examples, primary processing circuitry 50, telemetry circuitry 56, charging circuitry 68, or temperature sensor 59 correspond to individual hardware units, such as ASICs, DSPs, FPGAs, or other hardware units.

Memory 52 may store instructions that, when executed by primary processing circuitry 50, cause primary processing circuitry 50 and external charging device 208 to provide the functionality ascribed to external charging device 208 throughout this disclosure, or any equivalents thereof. For example, memory 52 may include instructions that cause primary processing circuitry 50 to control the power level used to charge IMD 210 in response to the determined temperatures for the housing/external surface(s) of IMD 210, as communicated from IMD 210, or instructions for any other functionality. Memory 52 may include a record of selected power levels, sensed temperatures, determined temperatures, or any other data related to charging rechargeable power source 18, described above in relation to FIG. 2. Memory 52 may store instructions that when executed by primary processing circuitry 50 may control the operation of indicator lights 64 as described above in relation to FIG. 1. Primary processing circuitry 50 may determine one or more operational states, e.g., of external charging device 208 and selectively control indicator lights 64 based on the operational state.

Primary processing circuitry 50 may, when requested, transmit any stored data in memory 52 to another computing device for review or further processing, such as to server 112 depicted in FIG. 1. Primary processing circuitry 50 may be configured to access memory, such as memory 32 of IMD 10 or memory 52 of external charging device 208, to retrieve information comprising instructions, formulas, and determined values for one or more constants.

User interface 54 may include buttons, such as control 62 or a keypad, lights, such as indicator lights 64, a speaker for voice commands, a display, such as a liquid crystal display (LCD), light-emitting diode (LED), or cathode ray tube (CRT). In some examples, the display may be a touch screen. Control 62 may be implemented as any type of component that may receive user input and provide an indication of the user input to primary processing circuitry 50. Control 62 may be a knob, switch, button and so on. As discussed in this disclosure, primary processing circuitry 50 may present and receive information relating to the charging or the status of rechargeable power source 18 (e.g., a battery) of IMD 210 via user interface 54. For example, user interface 54 may indicate when charging is occurring, quality of the alignment between internal primary coil 228 or external primary coil 48 and secondary coil 16 of IMD 210, the selected power level, current charge level of rechargeable power source 18, duration of the current recharge session, anticipated remaining time of the charging session, sensed temperatures, or any other information. Primary processing circuitry 50 may receive some of the information displayed on user interface 54 from IMD 210 in some examples. In some examples, user interface 54 may provide an indication to the user of the status of power source 18 of IMD 210. For example, user interface 54 may provide information indicative of the status of power source 18 (e.g., a battery status) including an indication of at least one of an amount (e.g., a percentage) of remaining charge, a time until recharge, a recharge interval, an expected date of battery depletion, or an expected date of battery recharge.

User interface 54 may also receive user input via user interface 54. The input may be, for example, in the form of pressing a button on a keypad or selecting an icon from a touch screen. The input may change programmed settings, start or stop therapy, request starting or stopping a recharge session, a desired level of charging, or one or more statistics related to charging rechargeable power source 18 (e.g., the cumulative thermal dose). In this manner, user interface 54 may allow the user to view information related to the operation of IMD 210. For example, control 62 may provide an input to primary processing circuitry 50 to cause primary processing circuitry 50 to start or stop delivery of wireless power to the power receiving device, e.g., IMD 10 or IMD 210 described above in relation to FIGS. 1 and 2.

Charging circuitry 58 may include one or more circuits that generate an electrical signal, and an electrical current, within external primary coil 48. Charging circuitry 58 may generate an alternating current of specified amplitude and frequency in some examples. In other examples, charging circuitry 58 may generate a direct current. In any case, charging circuitry 58 may be capable of generating electrical signals, and subsequent magnetic fields, to transmit various levels of power to IMD 210. In this manner, charging circuitry 58 may be configured to charge rechargeable power source 18 of IMD 210 with the selected power level.

Power source 60 may deliver operating power to the components of external charging device 208. Power source 60 may also deliver the operating power to drive external primary coil 48 during the charging process. Power source 60 may include a battery and a power generation circuit to produce the operating power. In some examples, a battery of power source 60 may be rechargeable to allow extended portable operation. In other examples, power source 60 may draw power from a wired voltage source such as a consumer or commercial power outlet.

Telemetry circuitry 56 supports wireless communication between IMD 210 and external charging device 208 under the control of primary processing circuitry 50. Telemetry circuitry 56 may also be configured to communicate with another computing device via wireless communication techniques, or direct communication through a wired connection. In some examples, telemetry circuitry 56 may be substantially similar to telemetry circuitry 36 of IMD 210 described herein, providing wireless communication via an RF or proximal inductive medium. In some examples, telemetry circuitry 56 includes an antenna 57, which may take on a variety of forms, such as an internal or external antenna. Although telemetry circuitry 56 and telemetry circuitry 36 may each include dedicated antennas for communications between these devices, telemetry circuitry 56 and telemetry circuitry 36 may instead, or additionally, be configured to utilize inductive coupling from internal primary coil 228 or external primary coil 48 to transfer data.

Examples of local wireless communication techniques that may be employed to facilitate communication between external charging device 208 and IMD 210 include radio frequency or inductive communication according to any of a variety of standard or proprietary telemetry protocols, or according to other telemetry protocols such as the Institute of Electrical and Electronics Engineers (IEEE) 802.11x or Bluetooth specification sets. In this manner, other external devices may be capable of communicating with external charging device 208 without needing to establish a secure wireless connection.

In operation, primary processing circuitry 50, or secondary processing circuitry 40, may control one or more sets of indicator lights 64 to provide information to a user about communication, charging efficiency, therapy status of the IMD and so on. For example, primary processing circuitry 50 may determine whether communication circuitry, e.g., telemetry circuitry 56, has established a communication link with a power receiving device (e.g., IMD 10 or IMD 210 depicted in FIGS. 1 and 2). Primary processing circuitry 50 may also determine whether the power receiving device (e.g., IMD 10 or IMD 210) is receiving wireless power, e.g., via charging circuitry 68 and internal primary coil 228, or charging circuitry 58 and external primary coil 48.

Primary processing circuitry 50 may use any one or more system metrics to determine power transfer to IMD 210. In some examples, IMD 210 may send a signal indicating an amount of current output by the recharge circuitry of IMD 210. In other examples, primary processing circuitry 50 may calculate other system metrics, such as alignment of internal primary coil 228 to secondary coil 16 of IMD 210 using any of several techniques, including heat calculations, temperature measurements, detection of metal, and so on. Primary processing circuitry 50 may compare any of the calculated power transfer, power efficiency, alignment, IMD 210 current, etc. to a threshold stored at memory 52. When above the threshold, primary processing circuitry 50 may cause indicator lights 64 to output a signal.

In some examples, primary processing circuitry 50 of external charging device 208 may be configured to determine the operational states of IMD 210. In some examples, primary processing circuitry 50 of external charging device 208 is configured to determine whether IMD 210 is in the first device state (e.g., when IMD 210 is configured to deliver therapy) or the second devices state (e.g., a deep sleep state). Primary processing circuitry 50 of external charging device 208 may determine the operational state of IMD 210 instead of or in addition to processing circuitry 30 of IMD 210 as described above. Further, primary processing circuitry 50 of external charging device 208 may be configured to perform any of the functions related to determining a status of power source 18 of IMD 210, as well as additionally or alternatively determining a status of power source 60 of external charging device 208.

In some examples, primary processing circuitry 50 may control haptic output circuitry 72 to provide a tactile sensation above the patient's perception level. For example, haptic output circuitry may vibrate or provide some similar tactile sensation. In some examples, primary processing circuitry 50 may control haptic output circuitry 72 to vibrate at a constant level for a specified duration, may output a pattern of vibration, or some similar haptic feedback for the patient. In some examples, the haptic feedback may indicate poor coupling, and the haptic feedback may fade as the coupling improves, e.g., the power receiving device is receiving wireless power above the first threshold. In this manner, the patient may receive feedback without the need to view user interface 54 of external charging device 208, or the user interface of some other device, e.g., a smart phone, tablet and so on. As discussed above, primary processing circuitry 50 may be configured to provide similar notifications or outputs related to the determination of the status of power source 18 of IMD 210. For example, primary processing circuitry 50 may control haptic output circuitry 72 to vibrate in a specific pattern to indicate to or alert the patient of the status of power source 18 of IMD 210.

FIG. 4 is a block diagram of an example programmer of FIG. 1. Programmer 204 may be a device for inputting information relating to a patient, receiving information from IMD 210, and updating IMD 210. In some examples, such as where programmer 204 is a patient programmer, programmer 204 can be a wearable communication device, with a therapy request input integrated into a key fob or a wristwatch, handheld computing device, smart phone, computer workstation, or networked computing device. Practically, programmer 204 can be a bring-your-own device provided by the patient, or provided by the healthcare provider in connection with the implantable device.

In some examples, such as where programmer 204 is physician/clinician programmer, programmer 204 is a tablet computing device that is preloaded with a specific application to interface with IMD 210. The physician or clinician may interact with programmer 204 for programming IMD 210. As described in more detail, the physician or clinician may utilize examples of a workflow to program IMD 210, as well as view information about the usage of IMD 210.

Programmer 204 generally comprises a processing circuitry 82, a memory 84, a user interface 86, communications circuitry 88, and a power source 90. Processing circuitry 82 can be any programmable device that accepts digital data as input, is configured to process the input according to instructions or algorithms, and provides results as outputs. In an example, processing circuitry 82 can be a central processing unit (CPU) configured to carry out the instructions of a computer program. Processing circuitry 82 is therefore configured to perform at least basic arithmetical, logical, and input/output operations. In one or more examples, processing circuitry 82 corresponds to individual hardware units, such as microprocessors, ASICs, DSPs, FPGAs, or other hardware units. In other examples, processing circuitry 82 can correspond to multiple individual hardware units, such as microprocessors, ASICs, DSPs, FPGAs, or other hardware units.

Memory 84 can comprise volatile or non-volatile memory as required by processing circuitry 82 to not only provide space to execute the instructions or algorithms, but to provide the space to store the instructions themselves. In one or more examples, volatile memory can include random access memory (RAM), dynamic random-access memory (DRAM), or static random access memory (SRAM), for example. In one or more examples, non-volatile memory can include read-only memory, flash memory, ferroelectric RAM, hard disk, floppy disk, magnetic tape, or optical disc storage, for example. The foregoing lists in no way limit the type of memory that can be used.

Furthermore, memory 84 may include, in certain embodiments, instructions 302, system inputs 400, system outputs 500, one or more machine learning algorithms 304, and one or more lookup tables 306. Furthermore, in certain embodiments, the instructions 302, the system inputs 400, the system outputs 500, the one or more machine learning algorithms 304, and the one or more lookup tables 306 could be stored locally on the programmer 204, externally on the server 112, or both.

The instructions 302 can be stored the memory 84 to dictate how the processing circuitry 82 performs one or more of the methods or functionalities described herein. In certain examples, the instructions 302 are stored in a form of non-volatile memory (such as flash memory, ROM, or EEPROM) that the processing circuitry 82 can read from. In certain embodiments, the instructions 302 are stored as binary data that is specific to the processing circuitry 82 of the system 100.

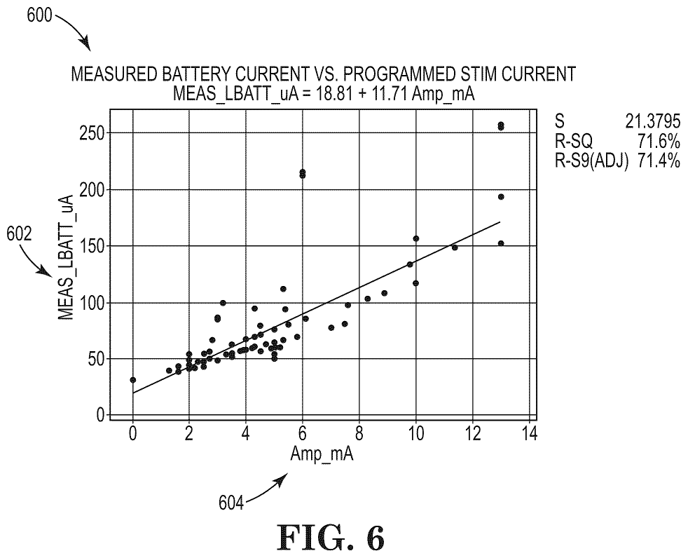

The system inputs 400 and the system outputs 500 include data that can be stored in a programmer memory 84 or externally on the server 112 (which is accessible via the communications circuitry 88). The system inputs 400 and the system outputs 500 are illustrated and described in further detail with respect to FIGS. 5-6.

The one or more machine learning algorithms 304 can be stored in the programmer memory 84 or externally on the server 112 to execute one or more of the methods described herein. A machine learning algorithm 304 is a computational model that allows the system 100 to learn patterns or make predictions based on data (e.g., the system inputs 400), without being explicitly programmed for every task. The one or more machine learning algorithms 304 analyze data, recognize patterns, and improve its performance over time as it processes additional information. A primary goal of machine learning is to enable systems to generalize from data, allowing them to make accurate predictions or decisions when faced with new, unseen data.

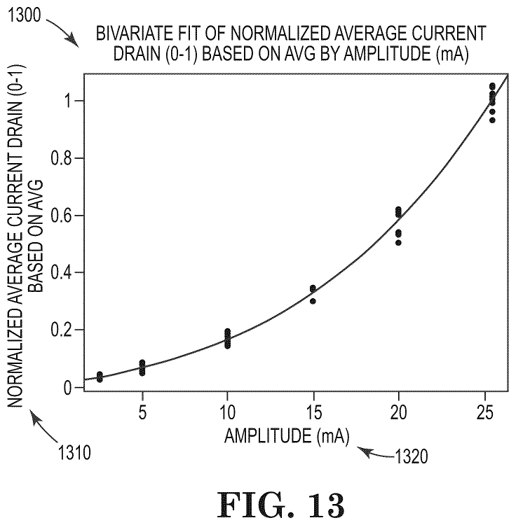

Machine learning can be categorized into two main types: supervised learning and unsupervised learning. In supervised learning, the one or more machine learning algorithms 304 are trained on by providing a labeled dataset (e.g., a labeled dataset corresponding to one or more IMDs), meaning that the input data is paired with correct output labels (e.g., an average predicted current drain of one or more batteries and an estimated run time of a battery of the IMD). The one or more machine learning algorithms 304 learn to map inputs to outputs (e.g., the system inputs 400 and the system outputs 500) by minimizing the difference between its predictions and the actual outcomes. This is commonly used for tasks like classification (e.g., identifying whether an email is spam or not) and regression (e.g., predicting house prices based on features like size and location). A process by which the one or more machine learning algorithms is trained using supervised learning is illustrated and described in further detail with respect to FIG. 13.

On the other hand, unsupervised learning involves training a model on data without predefined labels. The one or more machine learning algorithms 304 seek to identify hidden patterns or structures in the data, such as grouping similar data points into clusters (clustering) or reducing the dimensionality of complex datasets (dimensionality reduction). In certain embodiments, unsupervised learning is used to perform one or more of the methods discussed herein, including those illustrated and described in further detail with respect to FIG. 13.

In certain examples, the one or more lookup tables 306 can be stored in the programmer memory 84 or externally on the server 112 to execute one or more of the methods described herein. The one or more lookup tables 306 are illustrated and described in further detail with respect to FIGS. 5-13.

User interface 86 can include a button or keypad, lights, a speaker for voice commands, a knob able to turn, a display, such as a liquid crystal display (LCD), light-emitting diode (LED), or cathode ray tube (CRT). In some examples, the display may be a touch screen. Processing circuitry 82 can present and receive information relating to electrical stimulation and resulting therapeutic effects via user interface 86. For example, processing circuitry 82 can receive patient input via user interface 86. The input can be, for example, in the form of pressing a button on a keypad or selecting an icon from a touch screen. Processing circuitry 82 can also present information to the patient in the form of alerts related to delivery of the electrical stimulation to a patient or a caregiver via user interface 86. In some examples, user interface 86 may provide an indication to the user of the status of power source 18 of IMD 210. For example, user interface 86 may provide information indicative of the status of power source 18 (e.g., a battery status) including an indication of at least one of an amount (e.g., a percentage) of remaining charge, a time until recharge, a recharge interval, an expected date (e.g., including month, day, and year) of battery depletion, or an expected date (e.g., including month, day, and year) of battery recharge.

Communications circuitry 88 is configured to interface with IMD 210 and optionally, server 112 (FIG. 1). Communications circuitry 88 supports wireless communication between IMD 210 and, optionally, between server 112 and programmer 204 under the control of processing circuitry 82. Communications circuitry 88 can also be configured to communicate with another computing device via wireless communication techniques, or direct communication through a wired connection. Communications circuitry 88 can provide wireless communication via an RF or proximal inductive medium. In some examples, communications circuitry 88 can include an antenna, which may take on a variety of forms, such as an internal or external antenna.

Examples of local wireless communication techniques that may be employed to facilitate communication between programmer 204 and another computing device include RF communication according to the 802.11 or Bluetooth specification sets, infrared communication, e.g., according to the Infrared Data Association (IrDA) standard, or other standard or proprietary telemetry protocols. In this manner, other external devices may be capable of communicating with programmer 204 without needing to establish a secure wireless connection.

Power source 90 delivers operating power to the components of programmer 204. Power source 90 can include a battery and a power generation circuit to produce the operating power. In some examples, the battery may be rechargeable by, for example, an exterior power source.

Accordingly, as described, programmer 204 allows the user (e.g., patient, caretaker, clinician, physician) to program a therapy schedule and adjust therapy parameters (e.g., amplitude, frequency, or pulse width). A therapy schedule may include a frequency and duration of stimulation therapy based on certain time intervals (e.g., times of the day, amount of days between therapy sessions, particular dates or days of the week for therapy sessions, total duration or number of therapy sessions, etc.). Programmer 204 can communicate with IMD 210 to update the functionality of IMD 210.

In some examples, programmer 204 is configured to perform one or more of the functions related to determining a status of power source 18 (e.g., a battery status) of IMD 210, as described above. For example, programmer 204, via processing circuitry 82, may be configured to estimate of the status of power source 18 for one or more periods of time or one or more devices states of IMD 210. In some examples, programmer 204 is configured to track or estimate the status of power source 18 (e.g., a battery status) even when not connected to IMD 210. Programmer 204 may provide notifications related to an expected status of power source 18 of IMD 210. IMD 210 may provide updated information related to the status of power source 18 to programmer 204.

FIG. 5 is a block diagram illustrating example system inputs 400 that are received by the programmer of FIGS. 1 and 4. The system inputs can include, by non-limiting example and as shown in FIG. 5, one or more average predicted current drain parameters 410, one or more manufacturing parameters 430, and one or more other inputs 440.

The average predicted current drain parameters 410 can include one or more of the current amplitude A, the current pulse width P, the current rate R, a scheduled therapy duration D, and a scheduled therapy duty cycle X. The current amplitude A refers to a maximum strength of the electrical current flowing through the power source 90. Higher amplitudes can lead to increased energy consumption and heat generation, affecting the overall battery lifespan and performance. The current pulse width P is the duration of time that the current is applied during each pulse in a pulsed current operation. The current rate R denotes the speed at which current is drawn from the battery (often expressed in terms of how much charge is consumed over time). The scheduled therapy duration D is the total time a battery is used to power a specific therapeutic application or device. The scheduled therapy duty cycle X represents the ratio of the active time to the total time of operation, indicating how frequently the device is used.

FIG. 5 is a block diagram illustrating example system inputs 400 that are received by the programmer 204 or the server 112 of FIGS. 1 and 4, in accordance with one or more embodiments of the present disclosure. The inputs 400 can include, by non-limiting example, average predicted current drain parameters 410, manufacturing parameters 430, and other inputs 440.

The average predicted current drain parameters 410 can include one or more of the current amplitude A, the current pulse width P, the current rate R, a scheduled therapy duration D, and a scheduled therapy duty cycle X. The current amplitude A refers to a maximum strength of the electrical current flowing through the power source 90. Higher amplitudes can lead to increased energy consumption and heat generation, affecting the overall battery lifespan and performance. The current pulse width P is the duration of time that the current is applied during each pulse in a pulsed current operation. The current rate R denotes the speed at which current is drawn from the battery (often expressed in terms of how much charge is consumed over time). The scheduled therapy duration D is the total time a battery is used to power a specific therapeutic application or device. The scheduled therapy duty cycle X represents the ratio of the active time to the total time of operation, indicating how frequently the device is used.

In certain embodiments, one or more of the average predicted current drain can be changed to form a patient care plan that is unique to a patient. The current amplitude A may be increased if the patient is not experiencing a sufficient therapeutic effect with a current treatment plan. The current pulse width P can be increased to improve the stimulation's effectiveness, especially if the current amplitude alone is not providing adequate therapeutic results. The current rate may need to be increased if the patient requires a faster rate of stimulation for a desired therapeutic outcome. The scheduled therapy duration D may need to be increased if a longer period of stimulation is required to achieve therapeutic effects (e.g., to provide prolonged pain relief or prolonged neurostimulation). A scheduled therapy duty cycle X may need to be increased to provide more frequent or prolonged stimulation during each therapy session.

In certain embodiments, the manufacturing parameters 430 can include a quiescent current drain Q, a stimulation engine efficiency E, and other device information 432. The quiescent current drain Q refers to the current consumed by the implanted device when it is in a low-power, idle state, without performing active therapeutic functions. The stimulation engine efficiency E refers to the efficiency with which the implanted device's stimulation engine converts electrical energy into the desired therapeutic output (e.g., neurostimulation or muscle stimulation). In certain embodiments, the quiescent current drain Q and stimulation engine efficiency E are calculated using a stored history of telemetry sessions. In certain embodiments, the, the quiescent current drain Q and stimulation engine efficiency E are unique to the IMD based on one or more manufacturing parameters. In certain embodiments, the quiescent current drain (Q) and the stimulation engine efficiency (E) are calculated using a machine learning model.

The device information 432 can include one or more parameters that are unique to an IMD at the time of manufacture, including, but not limited to, at least one of a serial number, a lot number, a device model number, a manufacturing date, a software/firmware version, a device configuration, a unique device identifier, a batch or production code, or other implantation/usage instructions corresponding to the IMD. Furthermore, the device information 432 can include a battery type and capacity. In certain embodiments, the battery capacity has a minimum requirement of 10 milliampere-hours (mAh). In certain embodiments, the battery capacity has a distribution of 11.8 mAhs+/−0.6 mAhs.

Other inputs 440 can include one or more of telemetry time per day 442, other processing time per day 444, and therapy current drain choices 446. The telemetry time per day 442 refers to the amount of time each day that the IMD spends transmitting data to an external device, such as the programmer 204 or the server 112, to transmit patient data or receive commands. This may include, for example the time for each session (e.g., thirty minutes) and the number of sessions per week (e.g., between one to seven sessions per week). The other processing time per day 444 may include, by non-limiting example, time the IMD spends performing internal processing tasks that are unrelated to active therapy or telemetry, such as data logging, self-diagnostics, or background functions. The therapy current drain choices 446 may include, by non-limiting example, a therapy current drain and percentile choices of statistical measures for current consumption. In certain embodiments, the inputs may include a mean value (e.g., the average value of therapy current drain across all measurements or time points), a minimum value (e.g., the lowest observed therapy current drain during a measurement period), a maximum value (e.g., the highest observed therapy current drain during a measurement period), a tenth percentile value (e.g., a value below which 10% of the measurements fall, which measures a more conservative estimate of energy usage), a twenty-fifth percentile value (e.g., a value below which 25% of measurements fall, which measures a moderately conservative estimate of energy usage), a fiftieth percentile value (e.g., a value below which 50% of measurements fall, which measures a median value estimate of energy usage), a seventy-fifth percentile value (e.g., a value below which 75% of measurements fall, which measures a less conservative estimate of energy usage), and a ninetieth percentile value (e.g., a value below which 90% of measurements fall, which measures a more high-end estimate of energy usage). The therapy current drain choices 446 can be used to provide a range of estimates of when a battery may deplete based on the demands of a patient's therapy schedule.

In certain embodiments, the system 100 determines, based on at least one of the one or more average predicted current drain parameters 410, the manufacturing parameters 430, and other inputs 440, the average predicted current drain I_drain using a consumption model, the measured state of charge, and a predicted depletion time of the battery. This determination is illustrated and described in further detail with respect to the system outputs 500 of FIG. 6.

In certain embodiments, the system 100 may determine, based on a plurality of patient depletion schedules stored in a memory (e.g., memory 32, memory 52, programmer memory 84, or the server 112) a total predicted device run time for two or more depletion schedules. This may be useful, for example, in cases where one or more patients require varying depletion schedules that run consecutively.

FIG. 6 is a block diagram illustrating example system outputs 500 generated by the programmer 204 or the server 112 of FIGS. 1 and 5, in accordance with one or more embodiments of the present disclosure. In certain embodiments, the system outputs 500 can include an average predicted current drain I_drain, a predicted depletion time 502, and other user interface outputs 504.

In certain embodiments, the average predicted current drain 502 is a function of one or more of the average predicted current drain parameters 410 and the manufacturing parameters 430. In certain embodiments, the average predicted current drain I_drain is calculated using the equation I_drain=E*(A*P*R)*X+Q*(1−X) where E is the stimulation engine efficiency E, A is the current amplitude A, P is the current pulse width P, R is the current Rate R, X is the scheduled therapy duty cycle X, and Q is the quiescent current drain Q. In certain embodiments, the quiescent current drain Q and the stimulation engine efficiency E do not have to be calibrated in during the manufacturing process of each IMD (i.e., the quiescent current drain Q and the stimulation engine efficiency E are specific with respect to the IMD device information 432, such as the IMD manufacturing lot or an IMD model). In other examples, the quiescent current drain Q and the stimulation engine efficiency E can be calculated based on a number of scheduled therapy sessions over a time period 442 or a stored history of telemetry sessions.

In certain embodiments, the stimulation efficiency E is a function of parameters including the current amplitude A, the current pulse width P, and the current rate R. In some examples, by non-limiting example, the stimulation efficiency E could be a three-dimensional lookup table (e.g., lookup table 306) having the current amplitude A, the current pulse width P, and the current rate R being stored as the X, Y, and Z axes of the lookup table.

In certain embodiments, the average predicted current drain I_drain is calculated using the equation I_drain=((duty cycle X)*(load current+telemetry duty cycle)*(telemetry current+duty cycle for sleep mode)*(Power down current+duty cycle of other modes)*(background current))/100. Thus, this average predicted current drain calculation I_drain can be illustrated by the following equation:

$${I}_{Drain} = \frac{{duty}_{X} * {I}_{Load} + {duty}_{tel} * {I}_{Tel} + {duty}_{sleep} * {I}_{PPD} + {duty}_{other} * {I}_{Background}}{100}$$

In certain embodiments, one or more assumptions are made to simplify this equation. These assumptions may include, by non-limiting example, one or more of a power down current assumption (e.g., 130 μA), a background current assumption (e.g., 12 μA), a telemetry current assumption (e.g., 50 μA) and another background current assumption (e.g., 10.4 μA, which may include other processing steps of periodic switch closures for battery measurements and other firmware tasks).

The predicted depletion time 502 is determined by dividing the current state of charge (SOC) by the average predicted current drain I_drain. In certain embodiments, the system 100 calculates the current state of charge by utilizing one or more of a voltage measurement of the battery voltage or coulomb counting of a current flow into and out of the battery.

The user interface output(s) 504 include generating, for output to a user, the predicted depletion time of the IMD. This output may be shown on a user interface, such as one on the external charging device 208 (e.g., user interface 54) or the user interface of the programmer 204 (e.g., user interface 86).

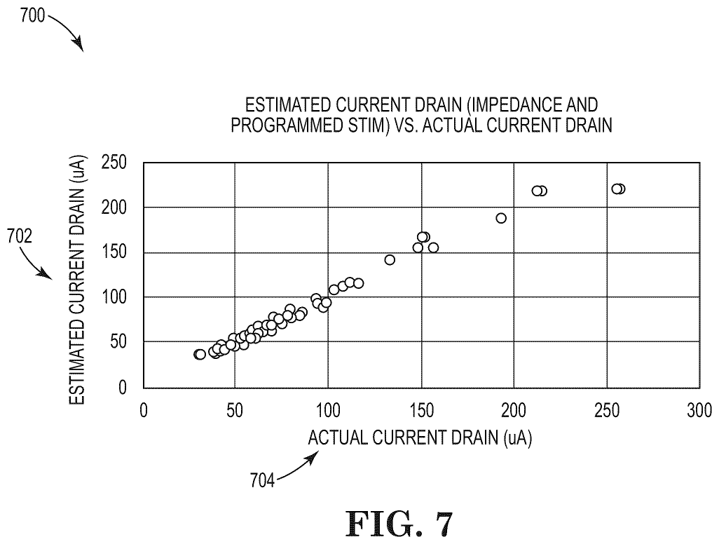

FIG. 7 is a scatter plot diagram 600 measuring an actual battery current 602 vs. a programmed stimulation current 604 using the system of FIG. 1, in accordance with one or more embodiments of the present disclosure.

Measuring the actual battery current 602 vs the programmed stimulation current 604 may aid in monitoring the efficiency of the battery (e.g., to ensure the measured current is not significantly higher than the programmed current, which might indicate inefficiencies or other processes that consume more power than expected). Further, measuring the actual battery current 602 vs the programmed stimulation current 604 may aid in battery life prediction by estimating an amount of current that is being drawn from the device against an expected current draw. As shown in FIG. 7, the actual battery current 602 increases steadily as the programmed stimulation increases, which indicates a relatively healthy battery in an IMD.

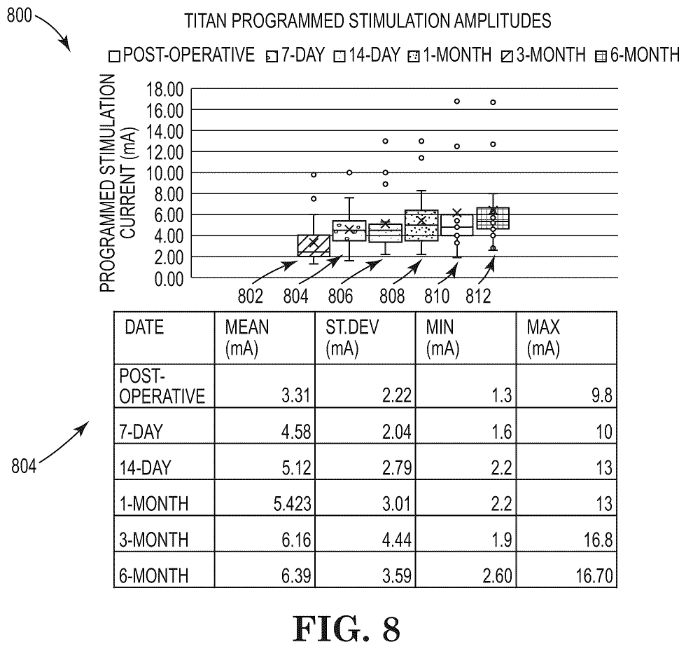

FIG. 8 is a scatter plot diagram 700 measuring an actual current drain of a battery 704 within an IMD vs. an estimated current drain of the battery 702 within the IMD using the system 100 of FIG. 1, in accordance with one or more embodiments of the present disclosure.

Similarly to what is illustrated and described with reference to FIG. 7, measuring an estimated current drain 702 vs the actual current drain 704 may aid in monitoring the efficiency of the battery (e.g., to ensure the measured current is not significantly higher than the programmed current, which might indicate inefficiencies or other processes that consume more power than expected). As shown in FIG. 8, the estimated current drain 702 increases steadily as the actual current drain 704 increases, which indicates a relatively healthy battery in an IMD limited inefficiencies.

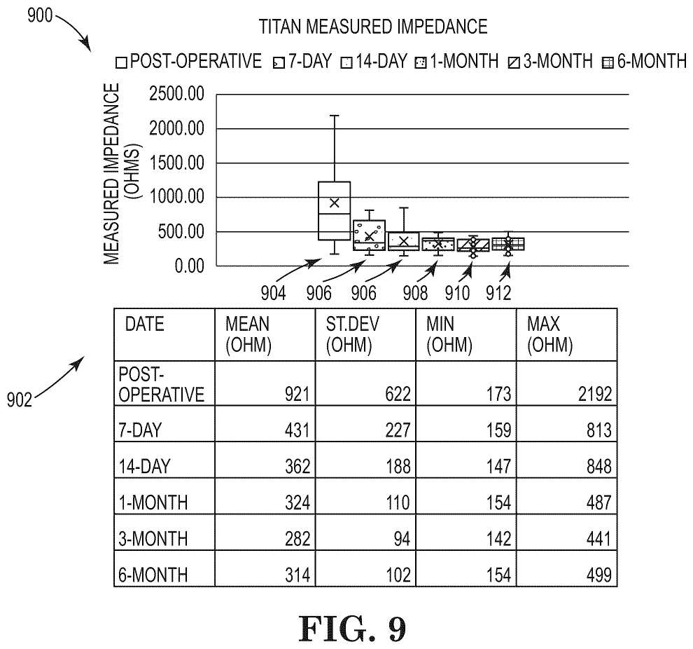

FIG. 9 is a diagram 800 measuring a programmed stimulation amplitude 800 of a battery within an IMD at different time intervals including post-operative programmed stimulation amplitude 802, a programmed stimulation amplitude measured seven days from implantation 804, a programmed stimulation amplitude measured fourteen days from implantation 804, a programmed stimulation amplitude measured one month from implantation 806, a programmed stimulation amplitude measured three months from implantation 808, and a programmed stimulation amplitude measured six months from implantation 810, in accordance with one or more embodiments of the present disclosure.

As illustrated in FIG. 9, and as shown in the diagram 800 of programmed stimulation current values 804, The stimulation amplitude increased over six months of collected data measured from a time after surgery (post operative 802).

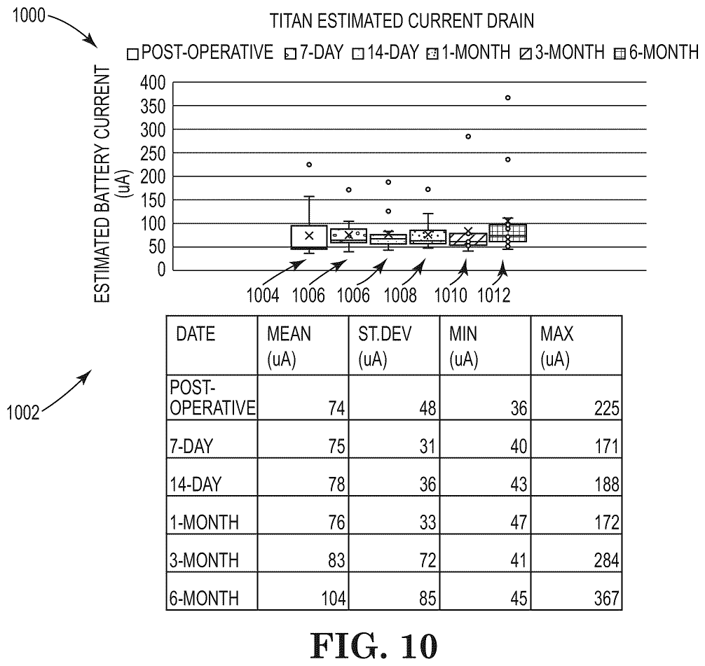

FIG. 10 is a diagram 900 measuring the impedance of a battery within an IMD at different time intervals including a post-operative impedance measurement 902, an impedance measurement seven days from implantation 904, an impedance measurement fourteen days from implantation 906, an impedance measurement one month from implantation 908, an impedance measurement three months from implantation 910, and an impedance measurement six months from implantation 912, in accordance with one or more embodiments of the present disclosure.

As illustrated in FIG. 10, and as shown in the diagram 900 of measured impedance values 804, The measured impedance decreased over six months of collected data measured from a time after surgery (post operative 902).

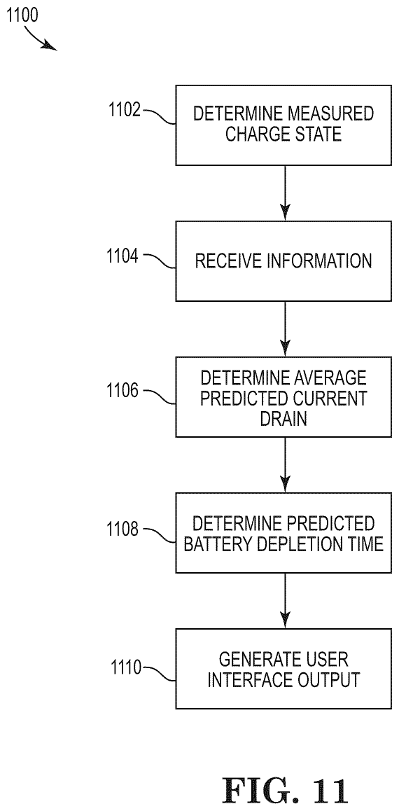

FIG. 11 is a diagram 1000 measuring an estimated current drain of a battery (also quantitatively shown in table 1002 below) within an IMD at different time intervals including post-operative estimated battery current 1004, an estimated battery current seven days from implantation 1006, an estimated battery current fourteen days from implantation 1008, an estimated battery current one month from implantation 1010, an estimated battery current three months from implantation 1012, and an estimated battery current six months from implantation 1014, in accordance with one or more embodiments of the present disclosure.

As illustrated in FIG. 11, the estimated battery current for patients remained relatively constant over six months of collected data measured from a time after surgery (post operative 1002).

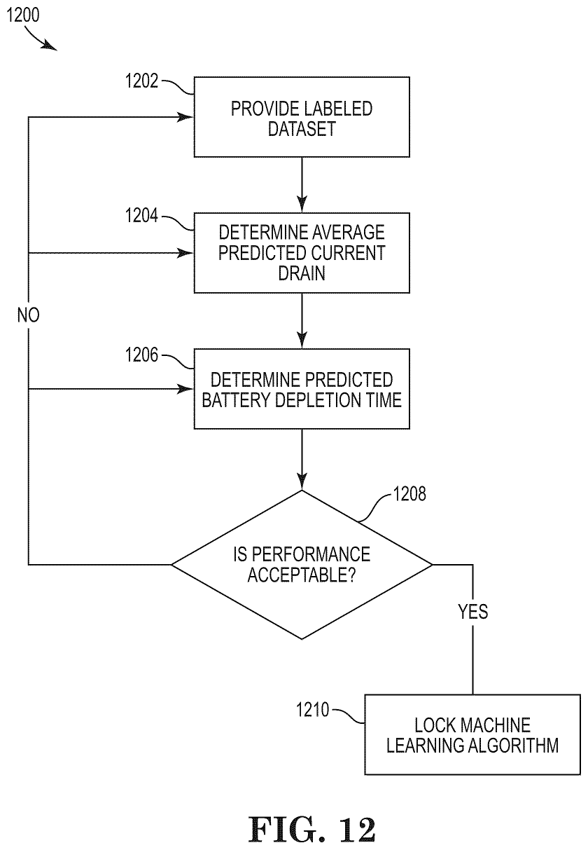

FIG. 12 is a flow chart diagram of a method 1100 for determining a predicted device run time of a battery and generating an output to a user including the predicted device run time, in accordance with one or more embodiments of the present disclosure.

The method 1100 includes a step 1102 of determining a measured charge state. As illustrated and described above with reference to FIGS. 3-6, in certain embodiments, the system 100 calculates the current state of charge by utilizing one or more of a voltage measurement of the battery voltage or coulomb counting of a current flow into and out of the battery.

The method 1100 includes a step 1104 of receiving information, including one or more system inputs 400. The system inputs 400 are illustrated and described in further detail with respect to FIG. 5. In certain embodiments, the system 100 may receive one or more system inputs that including the average predicted current drain parameters or the manufacturing parameters. The average predicted current drain parameters 410 may include, by non-limiting example, one or more of the current amplitude A, the current pulse width P, the current rate R, the scheduled therapy duration D, and the scheduled therapy duty cycle X. The manufacturing parameters 430 may include, by non-limiting example, the quiescent current drain Q, the stimulation engine efficiency E, and the device information 432.

The method 1100 includes a step 1106 of determining an average predicted current drain I_drain. The average predicted current drain I_drain can be calculated using the system inputs 400 FIG. 5. Further, one or more of the processes for calculating the current drain as illustrated and described in further detail with reference to FIGS. 3-6 can may be utilized.

The method 1100 includes a step 1108 of determining a predicted battery depletion time 1108. In certain embodiments, the predicted battery depletion time is calculated by dividing the determined state of charge (e.g., the measured charge state calculated in step 1102) by the average predicted current drain I_drain (e.g., the average predicted current drain I_drain calculated in step 1108). A method for determining a predicted battery depletion time is illustrated and described in further detail with respect to FIGS. 3-6.

The method 1100 includes a step 110 of generating an output to a user interface. As illustrated and described above with reference to FIGS. 3-6, the user interface output(s) 504 may be generated for output to a user to include the predicted depletion time of the IMD. This output may be shown on a user interface, such as one on the external charging device 208 (e.g., user interface 54) or the user interface of the programmer 204 (e.g., user interface 86).

FIG. 13 is a flow chart diagram of a method 1200 for training a machine learning algorithm to predict a depletion time of a battery within an IMD, in accordance with one or more embodiments of the present disclosure.

The method includes a step 1202 of providing a labeled dataset to the machine learning algorithm (e.g., machine learning algorithms 304 stored in at least one of the programmer 204 or server 112). In certain embodiments, the labeled dataset includes one or more of the system outputs 500 (e.g., an average predicted current drain I_drain, a predicted depletion time 502, an output for a user interface 504, and a determined state of charge). In certain embodiments, the labeled dataset can include one or more inputs 400. Furthermore, in certain embodiments, the labeled dataset can include one or more of the outputs 500 and the inputs 400.

Furthermore, in certain embodiments, the step 1202 may include receiving one or more manufacturing parameters 430 of the IMD, wherein the one or more manufacturing parameters include a quiescent current drain Q and a stimulation engine efficiency E and determining, based on the one or more manufacturing parameters 430 of the IMD and the one or more average predicted current drain parameters 410, the predicted device run time of the battery.

The method includes a step 1204 of determining an average predicted current drain I_drain. In certain embodiments, the average predicted current drain I_drain is calculated using one or more of the methods illustrated and described above in further detail with respect to FIGS. 3-6 and 12.

The method includes a step 1206 of determining a predicted battery depletion time 502. In certain embodiments, the predicted battery depletion time 502 is calculated using one or more of the methods illustrated and described above in further detail with respect to FIGS. 3-6 and 12.

The method includes a step 1208 of determining whether performance of the machine learning algorithm is acceptable when determining the average predicted current drain I_drain and the predicted battery depletion time 502.

In certain embodiments, step 1208 includes evaluating the machine learning algorithm using an accuracy evaluation function that compares the algorithm's predictions of the average predicted current drain I_drain and the predicted battery depletion time 502 to the one or more provided labels. In certain examples, if the machine learning algorithm achieves an accuracy that is above a predetermined threshold, the method 1200 may proceed to step 1210. In certain examples, if the machine learning algorithm does not achieve an accuracy that is above the predetermined threshold, then the method 1200 may repeat one or more of steps 1202, 1204, 1206.

In certain embodiments, the machine learning algorithm may evaluate its performance based on a desired precision, wherein the method 1200 proceeds to step 1210 if a threshold degree of precision is met and the method 1200 repeats one or more of steps 1202, 1204, 1206 if the threshold precision is not met.

In certain embodiments, the performance of the machine learning algorithm may evaluate its performance by any other validation process as recognizable by one of ordinary sill in the art.

The method 1200 includes a step 1210 of locking the machine learning algorithm for future use if the desired level of performance has been met (which is illustrated and described above in further detail with respect to step 1208).

A plurality of use cases are considered. In certain embodiments, a patient may receive an IMD and have follow up clinician programming or the patient may adjust therapy at home. In these embodiments, the patient or clinician may utilize the predicted battery depletion time outputted on the user interface to have an estimated date of when a battery of the IMD should be replaced or recharged. Furthermore, in certain embodiments, the patient or clinician may utilize the system 100 to update the predicted battery depletion time during a recharge session and predict whether recharging is needed prior to another recharge session that is scheduled in the future.

FIG. 13 illustrates a graph 1300 of normalized average current drain 1310 as a function of amplitude 1320. As shown in the graph 1300, system outputs 500 can be calculated using non-linear system inputs 400, including functions that include average predicted current drain parameters 410 that are not constrained to exhibit linearity; they may assume nonlinear forms, including higher-order exponents or other functional relationships.” For example, in certain embodiments, one or more system outputs 500 can be calculated by multiplying one or more average predicted current drain parameters 410 (which may include one or more of the current amplitude A, a current pulse width P, a current rate R, a scheduled therapy duration D, and a scheduled therapy duty cycle X) can exhibit a polynomial characteristics of first order, second order, third order, or any other order. Furthermore, in certain embodiments, a lookup table can be used to calculate relationships between one or more system inputs 400.

This disclosure includes the following non-limiting examples:

Example 1: a system comprising: processing circuitry configured to: determine a measured state of charge from a battery of an implantable medical device (IMD); receive information relating to an average predicted current drain (I_drain), wherein the information includes one or more average predicted current drain parameters, wherein the one or more average predicted current drain parameters include one or more of a current amplitude (A), a current pulse width (P), a current rate (R), a scheduled therapy duration (D), and a scheduled therapy session duty cycle (X); determine, based on the one or more average predicted current drain parameters, the average predicted current drain using a consumption model; determine, based on the measured state of charge and the average predicted current drain, a predicted depletion time of the battery; and generate, for output to a user, the predicted depletion time of the device.

Example 2: The system of example 1, wherein to determine the average predicted current drain, the processing circuitry is configured to: receive one or more manufacturing parameters of the IMD, wherein the one or more manufacturing parameters include a quiescent current drain (Q) or a stimulation engine efficiency (E); and determine, based on the one or more manufacturing parameters of the IMD and the one or more average predicted current drain parameters, the predicted device run time.

Example 3: The system of example 2, wherein: I_drain=E*(A*P*R)*X+Q*(1−X).

Example 4: The system of any of examples 2 to 3, wherein the quiescent current drain and the stimulation engine efficiency are specific with respect to an IMD manufacturing lot.

Example 5: The system of any of examples 2 to 4, wherein the quiescent current drain and the stimulation engine efficiency are specific with respect to an IMD model.

Example 6: The system of any of examples 2 to 5, wherein the stimulation engine efficiency (E) is a function of parameters including the current amplitude (A), the current pulse width (P), and the current rate (R).

Example 7: The system of example 6, wherein the current amplitude (A), the current pulse width (P), and the current rate (R) are stored within a three-dimensional lookup table.

Example 8: The system of any of examples 2 to 7, wherein the quiescent current drain (Q) and the stimulation engine efficiency (E) are calculated based on a number of scheduled therapy sessions over a time period.

Example 9: The system of any of examples 2 to 7, wherein the quiescent current drain (Q) and the stimulation engine efficiency (E) are calculated based on a stored history of telemetry sessions.

Example 10: The system of any of examples 2 to 9, wherein: the system includes a plurality of depletion schedules; and determining a total predicted device run time for two or more depletion schedules.

Example 11: The system of any of examples 2 to 10, wherein the quiescent current drain (Q) and the stimulation engine efficiency (E) are calculated using a machine learning model.

Example 12: A method comprising: determining a measured state of charge from a battery of an implantable medical device (IMD); receiving information relating to an average predicted current drain (I_drain), wherein the information includes one or more average predicted current drain parameters, wherein the one or more average predicted current drain parameters include one or more of a current amplitude (A), a current pulse width (P), a current rate (R), a scheduled therapy duration (D), and a scheduled therapy session duty cycle (X); determining, based on the one or more average predicted current drain parameters, the average predicted current drain using an empirical system model; determining, based on the measured state of charge and the average predicted current drain, a predicted device run time of the battery; and generating, for output to a user, the predicted device run time of the battery.

Example 13: The method of example 12, further comprising: receiving one or more manufacturing parameters of the IMD, wherein the one or more manufacturing parameters include a quiescent current drain (Q) and a stimulation engine efficiency (E); and determining, based on the one or more manufacturing parameters of the IMD and the one or more average predicted current drain parameters, the predicted device run time of the battery.

Example 14: The method of example 13, wherein, I_drain=E*(A*P*R)*X+Q*(1−X).

Example 15: The method of any of examples 13 to 14, further comprising determining the quiescent current drain and the stimulation engine efficiency based on a model of the IMD.

Example 16: The method of any of examples 13 to 15, further comprising: retrieving the current amplitude (A), the current pulse width (P), and the current rate (R) from a three-dimensional lookup table; and determining the stimulation engine efficiency (E) based on the current amplitude (A), the current pulse width (P), and the current rate (R).

Example 17: The method of any of examples 13 to 16, further comprising calculating the quiescent current drain (Q) and the stimulation engine efficiency (E) based on a history of telemetry sessions stored in a memory of the IMD.

Example 18: The method of any of examples 13 to 17, further comprising: determining a plurality of depletion schedules; and calculating a total predicted device run time by totaling the predicted device run time for each of the plurality of depletion schedules.

Example 19: A method for training a machine learning algorithm to predict a depletion time of a battery within an implantable medical device (IMD) comprising: providing a labeled dataset corresponding to one or more IMDs, wherein the labeled dataset includes, for each IMD: a measured state of charge from the battery; one or more average predicted current drain parameters comprising a current amplitude (A), a current pulse width (P), a current rate (R), a scheduled therapy duration (D), and a scheduled therapy session duty cycle (X); and a labeled depletion time of the battery; determining, based on the measured state of charge from the battery and the one or more average predicted current drain parameters, an average predicted current drain of one or more batteries; determining, based on the measured state of charge and the average predicted current drain, an estimated run time of the battery; evaluating the machine learning algorithm's performance on the labeled dataset by comparing the estimated run time of the battery to the labeled depletion time of the battery; and repeating one or more of the above steps until a desired level of accuracy is achieved.

Example 20: The method of example 19, further comprising: receiving one or more manufacturing parameters of the IMD, wherein the one or more manufacturing parameters include a quiescent current drain (Q) and a stimulation engine efficiency (E); and determining, based on the one or more manufacturing parameters of the IMD and the one or more average predicted current drain parameters, the predicted device run time of the battery.

Example 21: The method of any of examples 19-20, wherein the machine learning algorithm is trained using an unsupervised learning method.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112 (f) are not to be invoked unless the specific terms “means for” or “step for” are recited in a claim.

What is claimed is:

1. A system comprising:
processing circuitry configured to:
determine a measured state of charge from a battery of an implantable medical device (IMD);
receive information relating to an average predicted current drain (I_drain), wherein the information includes one or more average predicted current drain parameters, wherein the one or more average predicted current drain parameters include one or more of a current amplitude (A), a current pulse width (P), a current rate (R), a scheduled therapy duration (D), and a scheduled therapy session duty cycle (X);
determine, based on the one or more average predicted current drain parameters, the average predicted current drain using a consumption model;
determine, based on the measured state of charge and the average predicted current drain, a predicted depletion time of the battery; and
generate, for output to a user, the predicted depletion time of the IMD.

2. The system of claim 1, wherein to determine the average predicted current drain, the processing circuitry is configured to:
receive one or more manufacturing parameters of the IMD, wherein the one or more manufacturing parameters include a quiescent current drain (Q) or a stimulation engine efficiency (E); and
determine, based on the one or more manufacturing parameters of the IMD and the one or more average predicted current drain parameters, the predicted depletion time.

3. The system of claim 2, wherein:
$$I\_drain = E * ( A * P * R ) * X + Q * ( 1 - X ) .$$

4. The system of claim 2, wherein the quiescent current drain and the stimulation engine efficiency are specific with respect to an IMD manufacturing lot.

5. The system of claim 2, wherein the quiescent current drain and the stimulation engine efficiency are specific with respect to an IMD model.

6. The system of claim 2, wherein the stimulation engine efficiency (E) is a function of parameters including the current amplitude (A), the current pulse width (P), and the current rate (R).

7. The system of claim 6, wherein the current amplitude (A), the current pulse width (P), and the current rate (R) are stored within a three-dimensional lookup table.

8. The system of claim 2, wherein the quiescent current drain (Q) and the stimulation engine efficiency (E) are calculated based on a number of scheduled therapy sessions over a time period.

9. The system of claim 2, wherein the quiescent current drain (Q) and the stimulation engine efficiency (E) are calculated based on a stored history of telemetry sessions.

10. The system of claim 2, wherein:
the system includes a plurality of depletion schedules; and
forecasting when the battery will need to be recharged based on a plurality of therapy schedules.

11. The system of claim 2, wherein the quiescent current drain (Q) and the stimulation engine efficiency (E) are calculated using a machine learning model.

12. A method comprising:
determining a measured state of charge from a battery of an implantable medical device (IMD);
receiving information relating to an average predicted current drain (I_drain), wherein the information includes one or more average predicted current drain parameters, wherein the one or more average predicted current drain parameters include one or more of a current amplitude (A), a current pulse width (P), a current rate (R), a scheduled therapy duration (D), and a scheduled therapy session duty cycle (X);
determining, based on the one or more average predicted current drain parameters, the average predicted current drain using an empirical system model;
determining, based on the measured state of charge and the average predicted current drain, a predicted device run time of the battery; and
generating, for output to a user, the predicted device run time of the battery.

13. The method of claim 12, further comprising:
receiving one or more manufacturing parameters of the IMD, wherein the one or more manufacturing parameters include a quiescent current drain (Q) and a stimulation engine efficiency (E); and
determining, based on the one or more manufacturing parameters of the IMD and the one or more average predicted current drain parameters, the predicted device run time of the battery.

14. The method of claim 13, wherein:
$$I\_drain = E * ( A * P * R ) * X + Q * ( 1 - X ) .$$

15. The method of claim 13, further comprising determining the quiescent current drain and the stimulation engine efficiency based on a model of the IMD.

16. The method of claim 13, further comprising:
retrieving the current amplitude (A), the current pulse width (P), and the current rate (R) from a three-dimensional lookup table; and
determining the stimulation engine efficiency (E) based on the current amplitude (A), the current pulse width (P), and the current rate (R).

17. The method of claim 13, further comprising calculating the quiescent current drain (Q) and the stimulation engine efficiency (E) based on a history of telemetry sessions stored in a memory of the IMD.

18. The method of claim 13, further comprising:
determining a plurality of depletion schedules; and
calculating a total predicted device run time by totaling the predicted device run time for each of the plurality of depletion schedules.

19. A method for training a machine learning algorithm to predict a depletion time of a battery within an implantable medical device (IMD) comprising:
providing a labeled dataset corresponding to one or more IMDs, wherein the labeled dataset includes, for each IMD:
a measured state of charge from the battery;
one or more average predicted current drain parameters comprising a current amplitude (A), a current pulse width (P), a current rate (R), a scheduled therapy duration (D), and a scheduled therapy session duty cycle (X); and
a labeled depletion time of the battery;
determining, based on the measured state of charge from the battery and the one or more average predicted current drain parameters, an average predicted current drain of one or more batteries;
determining, based on the measured state of charge and the average predicted current drain, an estimated run time of the battery;
evaluating the machine learning algorithm's performance on the labeled dataset by comparing the estimated run time of the battery to the labeled depletion time of the battery; and
repeating one or more of the above steps until a desired level of accuracy is achieved.

20. The method of claim 19, further comprising:
receiving one or more manufacturing parameters of the IMD, wherein the one or more manufacturing parameters include a quiescent current drain (Q) and a stimulation engine efficiency (E); and
determining, based on the one or more manufacturing parameters of the IMD and the one or more average predicted current drain parameters, the predicted device run time of the battery.
